(19) United States
(12) Patent Application Publication
(10) Pub. No.: US 2003/0078231 A1
(43) Pub. Date: Apr. 24, 2003

(54) Orthomolecular sulpho-adenosylmethionine derivatives with antioxidant properties

(76) Inventor: Michael D. Wilburn, Cedar Hill, TX (US)

Correspondence Address: NATH & ASSOCIATES, 1030 15th STREET, 6TH FLOOR, WASHINGTON, DC, 20005, US

(21) Appl. No.: 09/886,612

(22) Filed: Jun. 22, 2001

(51) Int. Cl. A61K 31/7076; C07H 19/16

(52) U.S. Cl. 514/45; 536/27.3

(57) ABSTRACT

Orthomolecular Sulpho-Adenosylmethionine derivative compounds, compositions, and their uses for effecting a biological activity in an animal, such as neurochemical activity; liver biology activity; heart and artery function; cartilage, bone and joint health; stomach and/or intestinal lining resistance to ulceration; immune function; cell membrane integrity; and pain and inflammation. The compounds of the present invention are further useful for preventing or treating diseases or conditions; treating viral infections, infectious diseases, leukemia, and obesity; and reducing the risk of Sudden Infant Death Syndrome in an animal. The compounds of the present invention are of formula I:

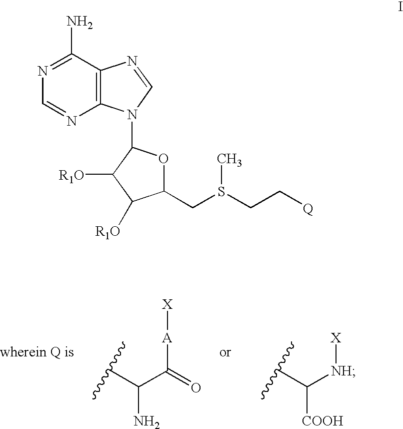

A is 0 or N; and

X is a reaction product as defined herein.

### BACKGROUND OF THE INVENTION

1. Field of Invention

The present invention relates to novel compounds and pharmaceutical preparations including the same which are useful in effecting a biological activity in an animal, such as neurochemical activity; liver biology activity; heart and artery function; cartilage, bone and joint health; stomach and/or intestinal lining resistance to ulceration; immune function; cell membrane integrity; and pain and inflammation; in preventing or treating diseases or conditions; in treating viral infections, infectious diseases, leukemia, and obesity; and in reducing the risk of Sudden Infant Death Syndrome.

2. Background

Linus Pauling coined the term “Orthomolecular Medicine” and defined it as: “The preservation of good health and the prevention and treatment of disease by varying the concentrations in the human body of the molecules or substances that are normally present, many of them required for life, such as the vitamins, essential amino acids, essential fats, and minerals.” Literally, the term is derived from the Greek “ortho”, for correct or right, and “molecule”, or “right molecule”. When these “right molecules” are out of balance, disorders and disease can result. The Orthomolecular Concomitant Theory of Convergence suggests that the duality of stress and uncontrolled free radical proliferation are major disruptive forces in the delicate balance of life, resulting in disorders, diseases, and premature death. In particular, the free radical theory of aging and disease suggests that excess free radicals can be generated by stress, simple aging, exposure to toxic pollutants in air, water, and foods, as well as cigarette smoke, alcohol, and ionizing radiation. Free radicals may produce oxidative damage to DNA and other cell components which accumulates with age and is suggested to be a major contributor to aging and degenerative diseases. Antioxidants may be used for reducing, eliminating, preventing, and reversing oxidative damage to tissues in an animal. Treatment of disorders and disease by orthomolecular methods is aimed at bringing such natural substances into healthful balance.

In a relatively recent phenomenon, traditional primary health care practitioners have begun to embrace orthomolecular nutrition as an enhancement to their practices. There are several forces promoting this trend, including consumer demand and the increasing eligibility of alternative health care for medical insurance coverage.

The National Institutes of Health (NIH) has established the National Center for Complementary and Alternative Medicine (NCCAM) to assist in prioritizing applications for research grants in complementary and alternative medicine (CAM). The NCCAM classification system is divided into seven major categories and includes examples of practices or preparations in each category. The biologically-based therapies category includes natural and biologically-based practices, interventions, and products. One subcategory is orthomolecular medicine, which refers to products used as nutritional and food supplements for preventive or therapeutic purposes. The NCCAM classification system lists Ascorbic Acid, Carotenes, Tocopherols, Folic Acid, Niacin, Niacinamide, Pantothenic Acid, Pyridoxine, Riboflavin, Thiamine, Vitamin A, Vitamin D, Vitamin K, Biotin, Choline, S-adenosylmethionine, Calcium, Magnesium, Selenium, Potassium, Taurine, Lysine, Tyrosine, Gamma-oryzanol, Iodine, Iron, Manganese, Molybdenum, Boron, Silicon, Vanadium, Co-enzyme Q₁₀, Carnitine, Probiotics, Glutamine, Phenylalanine, Glucosamine Sulfate, Chondroitin Sulfate, Lipoic Acid, Amino Acids, Phosphatidylserine, Melatonin, DHEA, Inositol, Glandular Products, Fatty Acids, and Medium Chain Triglycerides as examples of orthomolecular substances. Other examples of orthomolecular substances include Omega-3 fatty acids, lycopene, soy isoflavonoids, tocotrienols, chromium, zinc, and copper.

Sulpho-Adenosylmethionine, also known as S-adenosyl methionine or SAM, was first discovered in 1952 and has been commercially available in Europe since the 1970's. SAM appears to be an important compound that occurs in many living cells and takes part in several biological processes in the human body. U.S. Pat. No. 6,020,139 (Feb. 1, 2000) claims “a method to identify a therapeutic composition or protocol which ameliorates a disease or undesired condition in a subject, which method relies upon recognition of the existence of, and the interconnections between, eight SAM pathways” described therein and slates that “a new paradigm of disease centers around the metabolic pathways of S-adenosyl-L-methionine (SAM), the intermediates of these pathways and other metabolic pathways influenced by the SAM pathways.”

SAM is the key to at least three major biochemical pathways, including transmethylation, transsulfuration, and aminopropylation, which regulate or impact virtually every biochemical reaction in humans and animals (see FIG. 1). Neurochemistry; liver biology; heart and artery function; cartilage, bone, and joint health; stomach and intestinal lining resistance to ulceration; immune health; cell membrane integrity; pain and inflammation; resistance to viral infections and infectious diseases; response to leukemia; resistance to Sudden Infant Death Syndrome; and control of obesity are just some of the biological systems influenced by the efficiency or inefficiency of SAM metabolism. Pathways involving SAM also require a variety of vitamins and cofactors.

Transmethylation is essential to many biochemical processes: methyltransferase reactions shift a methyl group from a methyl donor molecule, such as SAM, to a methyl acceptor molecule (see FIG. 1), including biogenic amines (e.g. noradrenaline, serotonin), fatty acids and phospholipids, proteins, nucleic acids, polysaccharides, and porphyrins. SAM appears to be the most important methyl group donor in mammalian tissue. Methyl groups donated by SAM make possible the production of carnitine; acetyl-L-carnitine; creatine phosphate; adrenaline; phosphatidyl choline; and methyladenine and methylcytosine, among many other critical methylated biomolecules. Transmethylation byproduct homocysteine may be recycled via the methionine cycle to produce SAM (see FIGS. 1 and 2).

The transsulfuration pathway (see FIGS. 1 and 2) begins with transmethylation byproduct S-Adenosylhomocysteine (SAH). SAH is processed to yield homocysteine, which may be converted to taurine, sulfates, or cysteine. Cysteine may in turn produce a family of key sulphur compounds, including glutathione (GSH), GSH peroxidase, and GSH-S-transferase. SAM may also provide the sulphur for glucosamine sulphate and chondroitin sulphate. GSH, GSH compounds, and taurine play critical roles in liver detoxification of foreign and metabolic toxins. Failure to complete the transsulfuration pathway, or to recycle homocysteine via the methionine cycle, may result in the accumulation of homocysteine, which is toxic to heart and arterial tissues, and is a significant risk factor for cardio-pulmonary disorders.

Aminopropylation reactions utilizing SAM convert putrescine to spermidine and spermine (see FIG. 1), which play key roles in cell growth and differentiation, gene expression, and protein phosphorylation, calcium homeostasis, and neuronal regeneration. Methylthioadenosine is one byproduct of polyamine production and possesses potentially important analgesic and anti-inflammatory characteristics.

The novel compounds of the present invention have surprisingly been found to effect a variety of biological systems and processes, including neurochemical activity; liver biology activity; heart and artery function; cartilage, bone and joint health; stomach and/or intestinal lining resistance to ulceration; immune function; cell membrane integrity; pain and inflammation; resistance to viral infections and infectious diseases; response to leukemia; reducing the risk of Sudden Infant Death Syndrome; and control of obesity. Without being bound to a particular theory or mechanism of action, it appears that covalently linking SAM to a compound with antioxidant properties as disclosed herein may enhance the activity of SAM and/or the antioxidant compound in the relevant biochemical pathway.

Although others have recently recognized the significance of orthomolecular methods, Applicant is one of the pioneers in the field. In U.S. Pat. Nos. 5,108,754 (Apr. 28, 1992) and 5,177,208 (Jan. 5, 1993), Applicant obtained patent protection for novel compounds, compositions, and an orthomolecular method for treating sickle cell disease.

Other U.S. Patents for orthomolecular methods, as such, include the following: U.S. Pat. No. 4,500,515, for a method for treating alcohol and drug addicts; U.S. Pat. No.4,876,278, for zinc glycerolate complex and additions for pharmaceutical applications; U.S. Pat. Nos. 4,918,102 and 5,013,752, for prevention and treatment of alcoholism by the use of dietary chromium; U.S. Pat. Nos. 5,070,101 and 5,177,081, for a method and pharmaceutical composition for the treatment of schizophrenia; U.S. Pat. No. 5,230,996, for the use of ascorbate and tranexamic acid solution for organ and blood vessel treatment prior to transplantation; U.S. Pat. No. 5,278,189, for the prevention and treatment of occlusive cardiovascular disease with ascorbate and substances that inhibit the binding of lipoprotein (A) ; U.S. Pat. No. 5,869,525, for ascorbic acid drugs for intracerebral administration; U.S. Pat. Nos. 5,874,471 and 6,028,107, for the orthomolecular medical use of L-citrulline for vasoprotection, relaxative smooth muscle tone and cell protection; U.S. Pat. No. 5,897,891, for flavorful zinc compositions for oral use incorporating copper; and U.S. Pat. No. 6,039,978, for a dietary food enhancement agent.

Methods of producing stable SAM salts, both biologically and chemically (see U.S. Pat. Nos. 4,558,122, 4,621,056, 4,990,606, 5,102,791, 5,166,328, 5,114,931, and 5,128,249), are known in the art. Applicant has described a process for the synthesis of the SAM derivative α-(S-adenosylmethionine)-O-tocopherol in U.S. Pat. Nos. 5,108,754 and 5,177,208. All of the publications, patents, and other references cited in this application are incorporated by reference as though set forth in full.

Use of SAM alone is known in the art. Unexpectedly, it has been discovered that the novel compounds of the present invention may enhance the activity of SAM and/or a covalently-linked compound with antioxidant properties in the relevant biochemical pathway effecting neurochemical activity; liver biology activity; heart and artery function; cartilage, bone and joint health; stomach and/or intestinal lining resistance to ulceration; reducing the risk of Sudden Infant Death Syndrome; immune function; cell membrane integrity; and pain and inflammation in an animal. Further, such covalently linked conjugates are effective in preventing or treating diseases or conditions; and treating viral infections, infectious diseases, leukemia, and obesity in an animal.

All of the publications, patents, and other references cited herein are incorporated by reference as though set forth in full.

### SUMMARY OF THE INVENTION

The present invention relates to a novel Sulpho-Adenosylmethionine derivative compound of formula I:

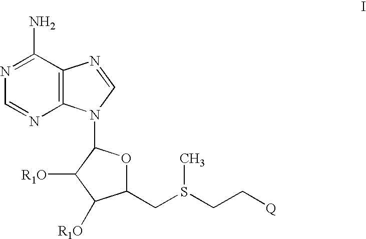

or a pharmaceutically acceptable salt, ester, or solvate, thereof, wherein:

R₁ is hydrogen, C₁-C₁₀ alkyl, C₂-C₁₀ alkenyl or alkynyl, or —C(O)R₂ where R₂ is C₁-C₁₀ alkyl or C₂-C₁₀ alkenyl or alkynyl;

Q is

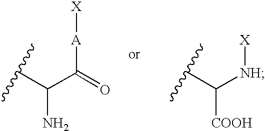

wherein

A is O or N; and

X is G, M, Y, or Z;

wherein G is a reaction product derived from a reactant compound selected from the group consisting of α-butyric acid, 3-methoxy-4-hydroxymandelic acid, 3-carboxy-3-aminopropyl analogues, e.g., wye base and diphthamide, 5-phosphoribose-1-pyrophosphoric acid, 6-gingerol, acetyl-L-carnitine, acetylcholine, ajoene, aminocyclopropane-carboxylic acid (ACC), anserine, anthocyanin, apigenin, arachidonic acid, astaxanthin, betaine, biopterin, calcium pectate, carbamyl phosphate, carnitine, carnosine, catechin, chlorogenic acid, choline, creatine, creatinine, cryptoxanthin, cumic acid, cumidine, curcumin, cyanidin chloride, d-limonene, daidzein, diacylglycerol, dopamine, ellagic acid, epicatechin, epicatechin gallate, epigallocatechin, epigallocatechin gallate, epinephrine, farnesyl, fibronectins, fisetin, (flavan-3-ol)ₙ, wherein n is 1-12, flavoxanthine, fructose 1,6-bisphosphate, gallic acid, genistein, geranyl, ginkgolide A, ginkgolide B, ginkgolide C, glucose, glutathione, GTP, hesperidin, hesperitin, histamine, HMG Co-A, homoserine lactone, indole-3-carbinol, kynurenine, L-dopa, L-histidine, linatine, lipoic acid, lupeol, lutein, luteolin, lycophyll, lycoxanthine, lysine, lysolecithin, mandelic acid, melanins, melatonin, metanephrine, methylated estrogen, methylated lipids, N-methylglycine, N-methyl histamine, N-malonyl ACC, neopterin, nervonic acid, N,N-dimethylglycine, N,N-dimethyltryptamine, norepinephrine, normetanephrine, ornithine, p-coumaric acid, pectin, phosphocreatine, phytic acid, phytochlorin, phytol, picolinic acid, proanthocyanin, pyruvate, quercetin, queuine, queuosine, quinolinic acid, rutin, S-allymercaptocysteine, sarcosine, serotonin, sesamin, silybin, sulphorane, taurine, taxicatin, taxicin I, taxicin II, taxifolin, taxine A, taxodione, tetrahydrobiopterin and derivatives, trimethylysine, tryptamine, tumeric, vaccenic acid, vanillic acid, xanthophyll, xanthoxylin, or zeaxanthin;

wherein M is a straight or branched C₁-C₁₁ alkyl, or C₂-C-₁₁ straight or branched alkenyl or alkynyl, wherein said alkyl, alkenyl, and alkynyl is substituted with 1 to 8 substituents selected from the group consisting of hydroxy, carboxy, amino, and —SH, wherein said hydroxy, carboxy, amino, or —SH substituent is optionally substituted; M is further optionally substituted with 1 to 8 substituents selected from the group consisting of halo, nitro, double-bonded oxygen, methoxy, and C₂-C₈ straight or branched alkoxy; and wherein one or more carbon atom(s) of said alkyl, alkenyl, or alkynyl is optionally replaced with nitrogen, oxygen, or sulfur;

wherein Y is a straight or branched C₁₂-C₃₀ alkyl, alkenyl, or alkynyl optionally substituted with 1 to 12 substituents selected from the group consisting of hydroxy, carboxy, amino, halo, nitro, —SH, and J, wherein J is phenyl or a 5-7 membered O-heterocyclic ring, and J is optionally substituted with 1 to 5 substituents selected from the group consisting of hydroxy, carboxy, amino, halo, nitro, —SH, C₁-C₁₀ alkyl C₂-C₁₀ alkenyl or alkynyl, methoxy, C₂-C₈ straight or branched alkoxy, and —OC(O)R₂ where R₂ is C₁-C₁₀ alkyl or C₂-C₁₀ alkenyl or alkynyl, wherein said hydroxy, carboxy, amino, halo, nitro, —SH, C₁-C₁₀ alkyl, C₂-C₁₀ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; and

wherein Z is phenyl substituted with 1 to 5 substituents selected from the group consisting of C₁-C₈ straight or branched alkyl, hydroxy, carboxy, amino, and —SH; and Z is further optionally substituted with 1 to 3 substituents selected from the group consisting of halo, nitro, C₁-C₈ straight or branched alkyl, C₂-C₈ straight or branched alkenyl or alkynyl, methoxy, and C₂-C₈ straight or branched alkoxy, wherein said hydroxy, carboxy, amino, —SH, halo, nitro, C₁-C₈ straight or branched alkyl, C₂-C₈ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur.

The present invention further relates to a method for preventing or treating diseases or conditions; effecting neurochemical activity; effecting liver biology activity; effecting heart and artery function; effecting cartilage, bone and joint health; effecting stomach and/or intestinal lining resistance to ulceration; effecting immune function; effecting cell membrane integrity; and effecting pain and inflammation in an animal, which comprises administering to said animal an effective amount of a Sulpho-Adenosylmethionine derivative compound of formula T.

Finally, the present invention relates to a pharmaceutical composition comprising:

(i) an effective amount of a Sulpho-Adenosylmethionine derivative compound of formula I; and

(ii) a pharmaceutically acceptable carrier.

### BRIEF DESCRIPTION OF THE DRAWINGS

FIG. 1 is a drawing which depicts three major metabolic pathways in which Sulpho-Adenosylmethionine (SAM) is a key factor: transmethylation, transsulfuration, and aminopropylation.

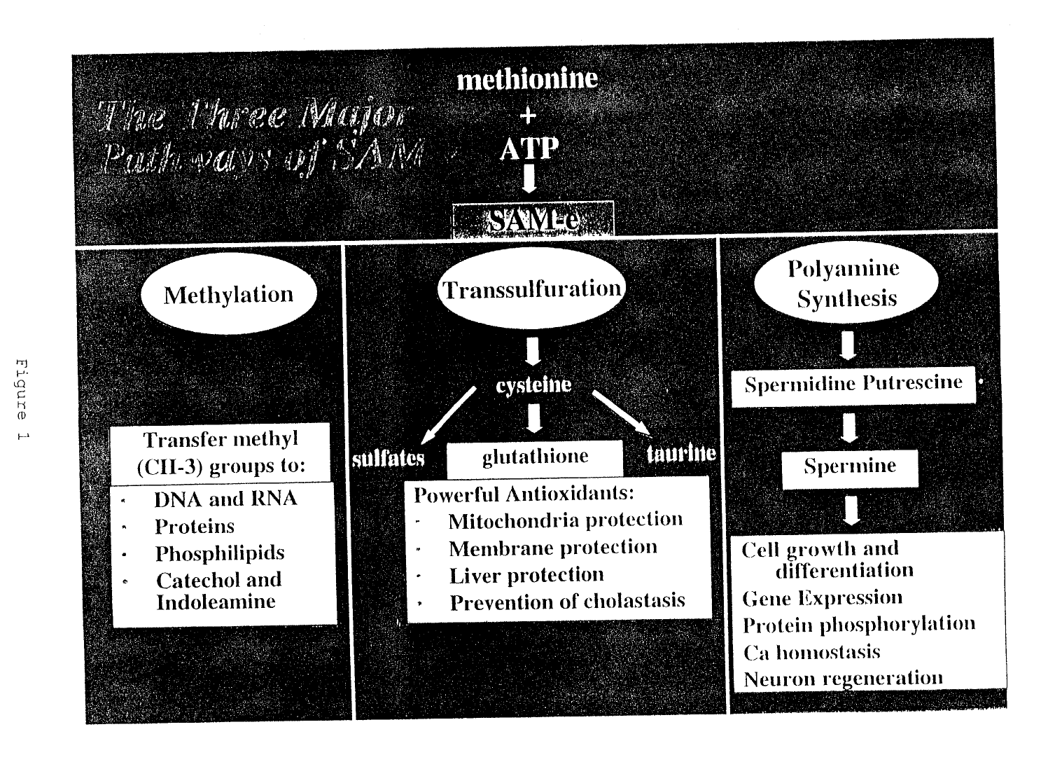

FIG. 2 is a drawing which depicts homocysteine metabolism via the methionine cycle and the transsulfuration pathway.

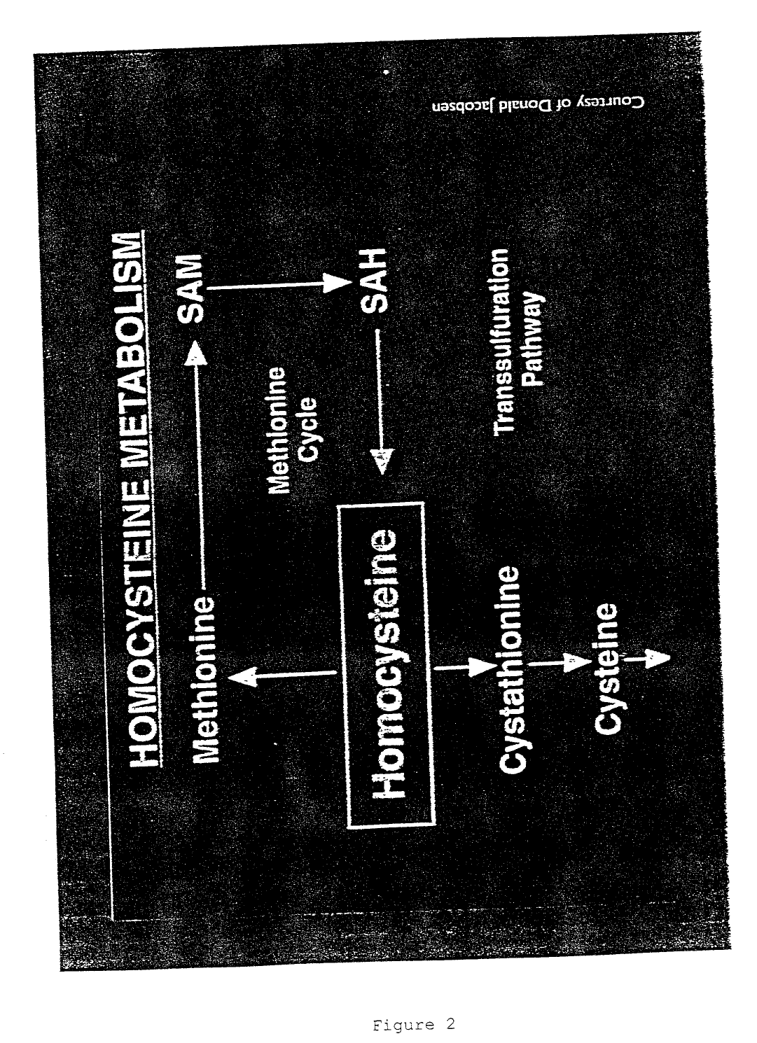

### DETAILED DESCRIPTION OF THE INVENTION

### DEFINITIONS

“Effecting” refers to the process of producing an effect on biological activity, function, health, or condition of an organism in which such biological activity, function, health, or condition is maintained, enhanced, diminished, or treated in a manner which is consistent with the general health and well-being of the organism.

“Enhancing” the biological activity, function, health, or condition of an organism refers to the process of augmenting, fortifying, strengthening, or improving.

“Isomers” refer to different compounds that have the same molecular formula. “Stereoisomers” are isomers that differ only in the way the atoms are arranged in space. “Enantiomers” are a pair of stereoisomers that are non-superimposable mirror images of each other. “Diastereoisomers” are stereolsomers which are not mirror images of each other. “Racemic mixture” means a mixture containing equal parts of individual enantiomers. “Non-racemic mixture” is a mixture containing unequal parts of individual enantiomers or stereoisomers.

“Pharmaceutically acceptable salt, ester, or solvate” refers to a salt, ester, or solvate of a subject compound which possesses the desired pharmacological activity and which is neither biologically nor otherwise undesirable. A salt, ester, or solvate can be formed with inorganic acids such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, gluconate, glycerophosphate, hemisulfate, heptanoate, hexanoate, hydrochloride, hydrobromide, hydroiodide, 2-hydroxyethanesulfonate, lactate, maleate, methanesulfonate, naphthylate, 2-naphthalenesulfonate, nicotinate, oxalate, sulfate, thiocyanate, tosylate and undecanoate. Examples of base salts, esters, or solvates include ammonium salts; alkali metal salts, such as sodium and potassium salts; alkaline earth metal salts, such as calcium and magnesium salts; salts with organic bases, such as dicyclohexylamine salts; N-methyl-D-glucamine; and salts with amino acids, such as arginine, lysine, and so forth. Also, the basic nitrogen-containing groups can be quarternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; aralkyl halides, such as benzyl and phenethyl bromides; and others. Water or oil-soluble or dispersible products are thereby obtained.

It is to be understood that, in its most common form, a “reactant compound” within the scope of the present invention may or may not have the reactive moiety(ies) necessary to produce a compound of the present invention. It is intended that such compound(s) will be derivatized to add one or more reactive moiety(ies) by means known to one of ordinary skill in the art. By way of example and not limitation, appropriate derivatives may be produced by hydration, halogenation, carboxylation, amination, nitration, and sulfonation.

“Reaction product” refers to that part of a reactant compound remaining after the chemical reaction producing a covalently-linked compound of the present invention. Such chemical reactions include substitution, elimination, addition, oxidation, and reduction reactions, and involve reactive moieties such as multiple bonds; oxygen and hydroxyl; nitrogen, nitro, amide, and amine; sulfur, sulfhydryl, and sulpho; and other common groups known to one of ordinary skill in the art.

“Sulpho-Adenosylmethionine”, or SAM, refers to a compound of the following formula:

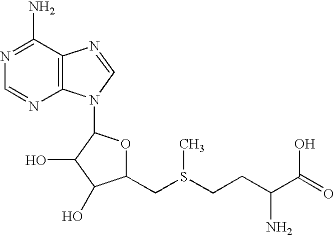

“Treating” refers to:

(i) preventing a disease and/or condition from occurring in a subject which may be predisposed to the disease and/or condition but has not yet been diagnosed as having it;

(ii) inhibiting the disease and/or condition, i.e., arresting its development; or

(iii) relieving the disease and/or condition, i.e., causing regression of the disease and/or condition.

### ORTHOMOLECULAR SULPHO-ADENOSYLMETHIONINE DERIVATIVES

The present invention relates to novel Sulpho-Adenosylmethionine derivative compounds of Formula I.

### Formula I

The Sulpho-Adenosylmethionine derivative may be a compound of formula I:

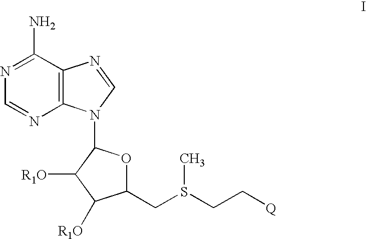

or a pharmaceutically acceptable salt, ester, or solvate, thereof, wherein:

R₁ Is hydrogen, C₁-C₁₀ alkyl, C₂-C₁₀ alkenyl or alkynyl, or —C(O)R₂ where R₂ is C₁-C₁₀ alkyl or C₂-C₁₀ alkenyl or alkynyl;

Q is

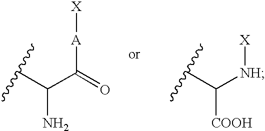

A is O or N; and

X is G, M, Y, or Z;

wherein G is a reaction product derived from a reactant compound selected from the group consisting of α-butyric acid, 3-methoxy-4-hydroxymandelic acid, 3-carboxy-3-aminopropyl analogues, e.g., wye base and diphthamide, 5-phosphoribose-1-pyrophosphoric acid, 6-gingerol, acetyl-L-carnitine, acetylcholine, ajoene, aminocyclopropane-carboxylic acid (ACC), anserine, anthocyanin, apigenin, arachidonic acid, astaxanthin, betaine, biopterin, calcium pectate, carbamyl phosphate, carnitine, carnosine, catechin, chlorogenic acid, choline, creatine, creatinine, cryptoxanthin, cumic acid, cumidine, curcumin, cyanidin chloride, d-limonene, daidzein, diacylglycerol, dopamine, ellagic acid, epicatechin, epicatechin gallate, epigallocatechin, epigallocatechin gallate, epinephrine, farnesyl, fibronectins, fisetin, (flavan-3-ol)ₙ, wherein n is 1-12, flavoxanthine, fructose 1,6-bisphosphate, gallic acid, genistein, geranyl, ginkgolide A, ginkgolide B, ginkgolide C, glucose, glutathione, GTP, hesperidin, hesperitin, histamine, HMG Co-A, homoserine lactone, indole-3-carbinol, kynurenine, L-dopa, L-histidine, linatine, lipoic acid, lupeol, lutein, luteolin, lycophyll, lycoxanthine, lysine, lysolecithin, mandelic acid, melanins, melatonin, metanephrine, methylated estrogen, methylated lipids, N-methylglycine, N-methyl histamine, N-malonyl ACC, neopterin, nervonic acid, N,N-dimethylglycine, N,N-dimethyltryptamine, norepinephrine, normetanephrine, ornithine, p-coumaric acid, pectin, phosphocreatine, phytic acid, phytochlorin, phytol, picolinic acid, proanthocyanin, pyruvate, quercetin, queuine, queuosine, quinolinic acid, rutin, S-allymercaptocysteine, sarcosine, serotonin, sesamin, silybin, sulphorane, taurine, taxicatin, taxicin I, taxicin II, taxifolin, taxine A, taxodione, tetrahydrobiopterin and derivatives, trimethylysine, tryptamine, tumeric, vaccenic acid, vanillic acid, xanthophyll, xanthoxylin, or zeaxanthin;

wherein M is a straight or branched C₁-C₁₁ alkyl, or C₂-C₁₁ straight or branched alkenyl or alkynyl, wherein said alkyl, alkenyl, and alkynyl is substituted with 1 to 8 substituents selected from the group consisting of hydroxy, carboxy, amino, and —SH, wherein said hydroxy, carboxy, amino, or —SH substituent is optionally substituted; M is further optionally substituted with 1 to 8 substituents selected from the group consisting of halo, nitro, double-bonded oxygen, methoxy, and C₂-C₈ straight or branched alkoxy; and wherein one or more carbon atom(s) of said alkyl, alkenyl, or alkynyl is optionally replaced with nitrogen, oxygen, or sulfur;

wherein Y is a straight or branched C₁₂-C₃₀ alkyl, alkenyl, or alkynyl optionally substituted with 1 to 12 substituents selected from the group consisting of hydroxy, carboxy, amino, halo, nitro, —SH, and J, wherein J is phenyl or a 5-7 membered O-heterocyclic ring, and J is optionally substituted with 1 to 5 substituents selected from the group consisting of hydroxy, carboxy, amino, halo, nitro, —SH, C₁-C₁₀ alkyl, C₂-C₁₀ alkenyl or alkynyl, methoxy, C₂-C₈ straight or branched alkoxy, and —OC(O)R₂ where R₂ is C₁-C₁₀ alkyl or C₂-C₁₀ alkenyl or alkynyl, wherein said hydroxy, carboxy, amino, halo, nitro, —SH, C₁-C₁₀ alkyl, C₂-C₁₀ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; and

wherein Z is phenyl substituted with 1 to 5 substituents selected from the group consisting of C₁-C₈ straight or branched alkyl, hydroxy, carboxy, amino, and —SH; and Z is further optionally substituted with 1 to 3 substituents selected from the group consisting of halo, nitro, C₁-C₈ straight or branched alkyl, C₂-C₈ straight or branched alkenyl or alkynyl, methoxy, and C₂-C₈ straight or branched alkoxy, wherein said hydroxy, carboxy, amino, —SH, halo, nitro, C₁-C₈ straight or branched alkyl, C₂-C₈ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur.

In a preferred embodiment, Q is (2,6-di(tert-butyl))4-methylphenol.

### METHODS OF THE PRESENT INVENTION

The present invention relates to a method for effecting neurochemical activity; effecting liver biology activity; effecting heart and artery function; effecting cartilage, bone and joint health; effecting stomach and/or intestinal lining resistance to ulceration; effecting immune function; effecting cell membrane integrity; and effecting pain and inflammation; preventing or treating diseases or conditions; treating viral infections; treating infectious diseases; treating leukemia; reducing the risk of Sudden Infant Death Syndrome; and treating obesity in an animal, which comprises administering to said animal an effective amount of a Sulpho-Adenosylmethionine derivative compound of formula I or formula II.

In a preferred embodiment, the disease(s) or condition(s) to be prevented or treated is/are selected from the group consisting of tissue damage resulting from physical trauma, tissue damage resulting from cell damage or cell death due to necrosis or apoptosis, neuronal mediated tissue damage or diseases, neural tissue damage resulting from ischemia and reperfusion injury, neurological disorders and neurodegenerative diseases, vascular stroke, cardiovascular disorders, age-related macular degeneration, AIDS and other immune diseases, arthritis, atherosclerosis, cachexia, cancer, degenerative diseases of skeletal muscle involving replicative senescence, diabetes, head trauma, immune senescence, inflammatory bowel disorders, muscular dystrophy, osteoarthritis, osteoporosis, chronic pain, acute pain, neuropathic pain, nervous insult, peripheral nerve injury, renal failure, retinal ischemia, septic shock, skin aging, altered circadian rhythmicicty, obesity, sickle cell anemia, cystic fibrosis, diseases or disorders relating to lifespan or proliferative capacity of cells, and diseases or disease conditions induced or exacerbated by cellular senescence.

In another preferred embodiment, the effect on neurochemical activity is selected from the group consisting of treating anxiety; treating depression; treating depression secondary to chronic diseases such as arthritis, fibromyalgia, liver disease, sickle cell disease, and alcoholism; treating dementia; treating schizophrenia; treating Alzheimer's disease; treating Parkinson's disease; treating demyelinating disorders; treating peripheral neuropathies; treating uremic neuropathy; treating Grand Mal seizures; treating Tay-Sachs disease; treating epilepsy; enhancing mood and behavior; and maintaining or effecting neuronal membrane ratios of phosphatidyl choline and cholesterol.

In another preferred embodiment, the effect on liver biology activity is selected from the group consisting of treating cirrhosis, chronic liver disease, alcoholic liver damage, toxic chemical exposure, non-steroidal anti-inflammatory drug-related liver damage, estrogen induced liver problems, bile disorders, and environmental chemical hypersensitivity.

In another preferred embodiment, the effect on heart and artery function is treating or reducing heart and/or artery disease risk due to elevated blood levels of homocysteine.

In a further preferred embodiment, the effect on cartilage, bone and joint health is selected from the group consisting of treating osteoarthritis, rheumatoid arthritis, fibromyalgia, joint injuries, joint inflammation, joint degeneration, and osteoporosis.

In another preferred embodiment, the effect on immune function is selected from the group consisting of treating organ transplant and graft rejection of non-self tissue, and treating autoimmune diseases involving the rejection of self tissue, such as lupus, uveitis, Behcet's disease, Graves disease, Guillain-Barre syndrome, psoriasis, acute dermatomyositis, atopic skin disease, scleroderma, eczema, aplastic anemia, primary cirrhosis, autoimmune hepatitis, ulcerative colitis, Crohn's disease, amyotrophic lateral sclerosis, myasthenia gravis, multiple sclerosis, nephrotic syndrome, glomerulonephritis, rheumatoid arthritis, and diabetes mellitus.

### PHARMACEUTICAL COMPOSITIONS OF THE PRESENT INVENTION

The present invention also relates to a pharmaceutical composition comprising:

(i) an effective amount of a Sulpho-Adenosylmethionine derivative compound of formula I ; and

(ii) a pharmaceutically acceptable carrier.

The novel pharmaceutical compositions of the invention include a therapeutically effective amount of the active agent indicated above. This effective amount will generally comprise from about 0.1 mg to about 100 mg of the active agent per kilogram of patient body weight per day. This effective amount can vary depending upon the physical status of the patient and other factors well known in the art. Moreover, it will be understood that this dosage of active agent can be administered in a single or multiple dosage units to provide the desired therapeutic effect. If desired, other therapeutic agents can be employed in conjunction with those provided by the present invention.

The compounds of the invention are preferably delivered to the patient by means of a pharmaceutically acceptable carrier. Such carriers are well known in the art and generally will be in either solid or liquid form. Solid form pharmaceutical preparations which may be prepared according to the present invention include powders, tablets, dispersible granules, capsules, cachets and suppositories. In general, solid form preparations will comprise from about 5% to about 90% by weight of the active agent.

A solid carrier can be one or more-substances which may also act as diluents, flavoring agents, solubilizers, lubricants, suspending agents, binders or tablet disintegrating agents; it can also be encapsulating material. In powders, the carrier is a finely divided solid which is in admixture with the viscous active compound. In tablets, the active compound is mixed with a carrier having the necessary binding properties in suitable proportions and compacted to the shape and size desired. Suitable solid carriers include magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating materials as a carrier which may provide a capsule in which the active component (with or without other carriers) is surrounded by carrier, which is thus in association with it. Similarly, cachets are included. Tablets, powders, cachets, and capsules can be used as solid dosage forms suitable for oral administration. If desired for reasons of convenience or patient acceptance, pharmaceutical tablets prepared according to the invention may be provided in chewable form, using techniques well known in the art.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby to solidify.

Liquid form preparations include solutions, suspensions, and emulsions. As an example may be mentioned water or water/propylene glycol solutions for parenteral injection. Liquid preparations can also be formulated in solution in aqueous polyethylene glycol solution. Aqueous solutions suitable for oral use can be prepared by dissolving the active component in water and adding suitable colorants, flavors, stabilizers and thickening agents as desired. Aqueous suspensions suitable for oral use can be made my dispersing the finely divided active component in water with a viscous material, i.e., natural or synthetic gums, resins, methylcellulose, sodium carboxymethylcellulose, and other well known suspending agents. Liquid pharmaceutical preparations may comprise up to 100% by weight of the subject active agent.

Also contemplated as suitable carriers are solid form preparations which are intended to be converted, shortly before use, to liquid form preparations for either oral or parenteral administration. Such liquid forms include solutions, suspensions, and emulsions. These particular solid form preparations are most conveniently provided in unit dose form and as such are used to provide a single liquid dosage unit. Alternately, sufficient solid may be provided so that after conversion to liquid form, multiple individual liquid doses may be obtained by measuring predetermined volumes of the liquid form preparation as with a syringe, teaspoon, or other volumetric container. When multiple liquid doses are so prepared, it is preferred to maintain the unused portion of said liquid doses at low temperature (i.e., under refrigeration) in order to retard possible decomposition. The solid form preparations intended to be converted to liquid form may contain, in addition to the active material, flavorants, colorants, stabilizers, buffers, artificial and natural sweeteners, dispersants, thickeners, solubilizing agents, and the like. The liquid utilized for preparing useful liquid form preparations may be water, isotonic water, ethanol, glycerine, propylene glycol, and the like as well as mixtures thereof. Naturally, the liquid utilized will be chosen with regard to the route of administration. For example, liquid preparations containing large amounts of ethanol are not suitable for parenteral use.

The pharmaceutical preparation may also be in a unit dosage form. In such form, the preparation is subdivided into unit doses containing appropriate quantities of the active component. The unit dosage form can be a packaged preparation, the package containing discrete quantities of preparation, for example, packeted tablets, capsules, and powders in vials or ampoules. The unit dosage form can also be a capsule, cachet, or tablet itself or it can be the appropriate number of any of these in packaged form.

The pharmaceutical preparations of the invention may include one or more preservatives well known in the art, such as benzoic acid, sorbic acid, methylparaben, propylparaben and ethylenediaminetetraacetic acid (EDTA). Preservatives are generally present in amounts up to about 1% and preferably from about 0.05 to about 0.5% by weight of the pharmaceutical composition.

Useful buffers for purposes of the invention include citric acid-sodium citrate, phosphoric acid-sodium phosphate, and acetic acid-sodium acetate in amounts up to about 1% and preferably from about 0.05 to about 0.5% by weight of the pharmaceutical composition. Useful suspending agents or thickeners include cellulosics like methylcellulose, carageenans like alginic acid and its derivatives, xanthan gums, gelatin, acacia, and microcrystalline cellulose in amounts up to about 20% and preferably from about 1% to about 15% by weight of the pharmaceutical composition.

Sweeteners which may be employed include those sweeteners, both natural and artificial, well known in the art. Sweetening agents such as monosaccharides, disaccharides and polysaccharides such as xylose, ribose, glucose, mannose, galactose, fructose, dextrose, sucrose, maltose, partially hydrolyzed starch or corn syrup solids and sugar alcohols such as sorbitol, xylitol, mannitol and mixtures thereof may be utilized in amounts from about 10% to about 60% and preferably from about 20% to about 50% by weight of the pharmaceutical composition. Water soluble artificial sweeteners such as saccharin and saccharin salts such as sodium or calcium, cyclamate salts, acesulfame-K, aspartame and the like and mixtures thereof may be utilized in amounts from about 0.001% to about 5% by weight of the composition.

Flavorants which may be employed in the pharmaceutical products of the invention include both natural and artificial flavors, and mints such as peppermint, menthol, vanilla, artificial vanilla, chocolate, artificial chocolate, cinnamon, various fruit flavors, both individually and mixed, in amounts from about 0.5% to about 5% by weight of the pharmaceutical composition.

Colorants useful in the present invention include pigments which may be incorporated in amounts of up to about 6% by weight of the composition. A preferred pigment, titanium dioxide, may be incorporated in amounts up to about 1%. Also, the colorants may include other dyes suitable for food, drug and cosmetic applications, known as F.D.&C. dyes and the like. Such dyes are generally present in amounts up to about 0.25% and preferably from about 0.05% to about 0.2% by weight of the pharmaceutical composition. A full recitation of all F.D.&C. and D.&C. dyes and their corresponding chemical structures may be found in the Kirk-Othmer Encyclopedia of Chemical Technology, in Volume 5, at pages 857-884, which text is accordingly incorporated herein by reference.

Useful solubilizers include alcohol, propylene glycol, polyethylene glycol and the like and may be used to solubilize the flavors. Solubilizing agents are generally present in amounts up to about 10%; preferably from about 2% to about 5% by weight of the pharmaceutical composition.

Lubricating agents which may be used when desired in the instant compositions include silicone oils or fluids such as substituted and unsubstituted polysiloxanes, e.g., dimethyl polysiloxane, also known as dimethicone. Other well known lubricating agents may be employed.

It is not expected that compounds of the present invention will display significant adverse interactions with other synthetic or naturally occurring substances. Thus, a Sulpho-Adenosylmethionine derivative compound of the present invention may be administered in combination with other compounds and compositions useful for effecting neurochemical activity; liver biology activity; heart and artery function; cartilage, bone and joint health; stomach and/or intestinal lining resistance to ulceration; immune function; cell membrane integrity; and pain and inflammation; treating viral infections, infectious diseases, leukemia, and obesity; and reducing the risk of Sudden Infant Death Syndrome. In particular the compounds of the present invention may be administered in combination with other compounds of the present invention; other orthomolecular substances; vitamin(s) and/or cofactor(s) selected from the group consisting of Vitamin A, β-Carotene, Vitamin B₁ (Thiamine), Vitamin B₂ (Riboflavin), Vitamin B₆ (Pyrodoxine), Biotin, Inositol, Folic Acid, Vitamin B₁₂ (Cyanocobalamine), Nicotinic Acid, Vitamin C (esterified or non-esterified Ascorbic Acid), Vitamin D (Ergocalciferol), Pantothenic Acid, Phosphatidyl Choline, Tetrahydrofolate, Tetrahydrobiopterin, β-tocopherol, γ-tocopherol, δ-tocopherol, α-tocotrienol, β-tocotrienol, γ-tocotrienol, δ-tocotrienol, Para-aminobenzoic Acid (PABA), and Inosinate; other known over-the-counter and/or prescription drugs; and other compounds and compositions useful for effecting neurochemical activity; liver biology activity; heart and artery function; cartilage, bone and joint health; stomach and/or intestinal lining resistance to ulceration; immune function; cell membrane integrity; and pain and inflammation; treating viral infections, infectious diseases, leukemia, and obesity; and reducing the risk of Sudden Infant Death Syndrome.

The optimal pharmaceutical formulations will be determined by one skilled in the art depending upon considerations such as the route of administration and desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the present therapeutic agents of the invention.

### Synthesis of Sulpho-Adenosylmethionine Derivatives

The compounds of the present invention may be readily prepared by standard techniques of organic chemistry, utilizing the general synthetic pathways depicted below.

In the preparation of the compounds of the invention, one skilled in the art will understand that one may need to protect or block various reactive functionalities on the starting compounds or intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry. One skilled in the art is referred to “Protective Groups in Organic Chemistry,” McOmie, ed., Plenum Press, New York, N.Y.; and “Protective Groups in Organic Synthesis,” Greene, ed., John Wiley & Sons, New York, N.Y. (1981) for the teaching of protective groups which may be useful in the preparation of compounds of the present invention.

The product and intermediates may be isolated or purified using one or more standard purification techniques, including, for example, one or more of simple solvent evaporation, recrystallization, distillation, sublimation, filtration, chromatography, including thin-layer chromatography, HPLC (e.g. reverse phase HPLC), column chromatography, flash chromatography, radial chromatography, trituration, and the like.

### SCHEME I

As depicted by Scheme I, a radical of α-butyric acid, 3-methoxy-4-hydroxymandelic acid, 3-carboxy-3-aminopropyl analogues, e.g., wye base and diphthamide, 5-phosphoribose-1-pyrophosphoric acid, 6-gingerol, acetyl-L-carnitine, acetylcholine, ajoene, aminocyclopropane-carboxylic acid (ACC), anserine, anthocyanin, apigenin, arachidonic acid, astaxanthin, betaine, biopterin, calcium pectate, carbamyl phosphate, carnitine, carnosine, catechin, chlorogenic acid, choline, creatine, creatinine, cryptoxanthin, cumic acid, cumidine, curcumin, cyanidin chloride, d-limonene, daidzein, diacylglycerol, dopamine, ellagic acid, epicatechin, epicatechin gallate, epigallocatechin, epigallocatechin gallate, epinephrine, farnesyl, fibronectins, fisetin, (flavan-3-ol)ₙ, wherein n is 1-12, flavoxanthine, fructose 1,6-bisphosphate, gallic acid, genistein, geranyl, ginkgolide A, ginkgolide B, ginkgolide C, glucose, glutathione, GTP, hesperidin, hesperitin, histamine, HMG Co-A, homoserine lactone, indole-3-carbinol, kynurenine, L-dopa, L-histidine, linatine, lipoic acid, lupeol, lutein, luteolin, lycophyll, lycoxanthine, lysine, lysolecithin, mandelic acid, melanins, melatonin, metanephrine, methylated estrogen, methylated lipids, N-methylglycine, N-methyl histamine, N-malonyl ACC, neopterin, nervonic acid, N,N-dimethylglycine, N,N-dimethyltryptamine, norepinephrine, normetanephrine, ornithine, p-coumaric acid, pectin, phosphocreatine, phytic acid, phytochlorin, phytol, picolinic acid, proanthocyanin, pyruvate, quercetin, queuine, queuosine, quinolinic acid, rutin, S-allymercaptocysteine, sarcosine, serotonin, sesamin, silybin, sulphorane, taurine, taxicatin, taxicin I, taxicin II, taxifolin, taxine A, taxodione, tetrahydrobiopterin and derivatives, trimethylysine, tryptamine, tumeric, vaccenic acid, vanillic acid, xanthophyll, xanthoxylin, or zeaxanthin bearing a free hydroxyl (from —OH or —COOH) or amine functional group may be covalently attached to S-adenosylmethionine to produce a compound of formula I:

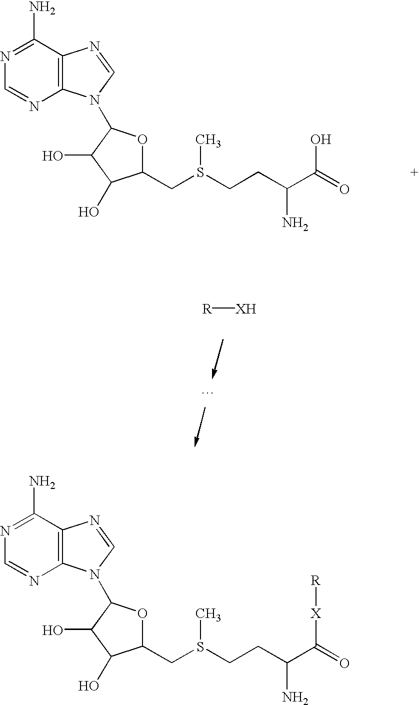

wherein X is 0 or NH.

### SCHEME II

As depicted by Scheme II, a radical of α-butyric acid, 3-methoxy-4-hydroxymandelic acid, 3-carboxy-3-aminopropyl analogues, e.g., wye base and diphthamide, 5-phosphoribose-l-pyrophosphoric acid, 6-gingerol, acetyl-L-carnitine, acetylcholine, ajoene, aminocyclopropane-carboxylic acid (ACC), anserine, anthocyanin, apigenin, arachidonic acid, astaxanthin, betaine, biopterin, calcium pectate, carbamyl phosphate, carnitine, carnosine, catechin, chlorogenic acid, choline, creatine, creatinine, cryptoxanthin, cumic acid, cumidine, curcumin, cyanidin chloride, d-limonene, daidzein, diacylglycerol, dopamine, ellagic acid, epicatechin, epicatechin gallate, epigallocatechin, epigallocatechin gallate, epinephrine, farnesyl, fibronectins, fisetin, (flavan-3-ol)ₙ, wherein n is 1-12, flavoxanthine, fructose 1,6-bisphosphate, gallic acid, genistein, geranyl, ginkgolide A, ginkgolide B, ginkgolide C, glucose, glutathione, GTP, hesperidin, hesperitin, histamine, HMG Co-A, homoserine lactone, indole-3-carbinol, kynurenine, L-dopa, L-histidine, linatine, lipoic acid, lupeol, lutein, luteolin, lycophyll, lycoxanthine, lysine, lysolecithin, mandelic acid, melanins, melatonin, metanephrine, methylated estrogen, methylated lipids, N-methylglycine, N-methyl histamine, N-malonyl ACC, neopterin, nervonic acid, N,N-dimethylglycine, N,N-dimethyltryptamine, norepinephrine, normetanephrine, ornithine, p-coumaric acid, pectin, phosphocreatine, phytic acid, phytochlorin, phytol, picolinic acid, proanthocyanin, pyruvate, quercetin, queuine, queuosine, quinolinic acid, rutin, S-allymercaptocysteine, sarcosine, serotonin, sesamin, silybin, sulphorane, taurine, taxicatin, taxicin I, taxicin II, taxifolin, taxine A, taxodione, tetrahydrobiopterin and derivatives, trimethylysine, tryptamine, tumeric, vaccenic acid, vanillic acid, xanthophyll, xanthoxylin, or zeaxanthin, bearing a free hydroxyl (from —OH or —COOH) or amine functional group may be covalently attached to S-adenosylmethionine to produce a compound of formula II:

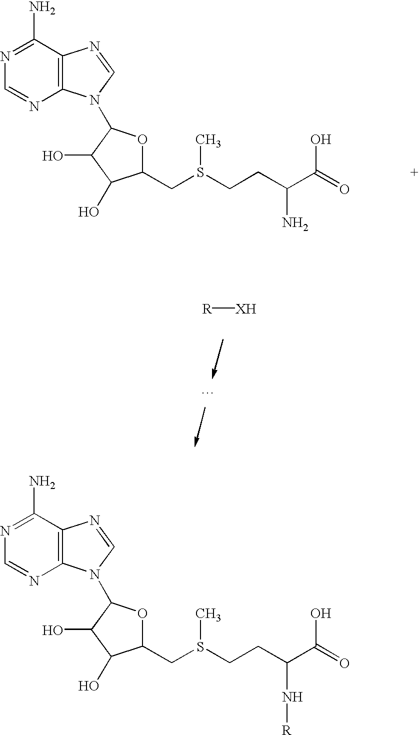

wherein X is O or NH.

### SCHEME III

A representative example of the synthesis of a compound of formula I, covalently linking a radical of curcumin with S-adenosylmethionine, is depicted in Scheme

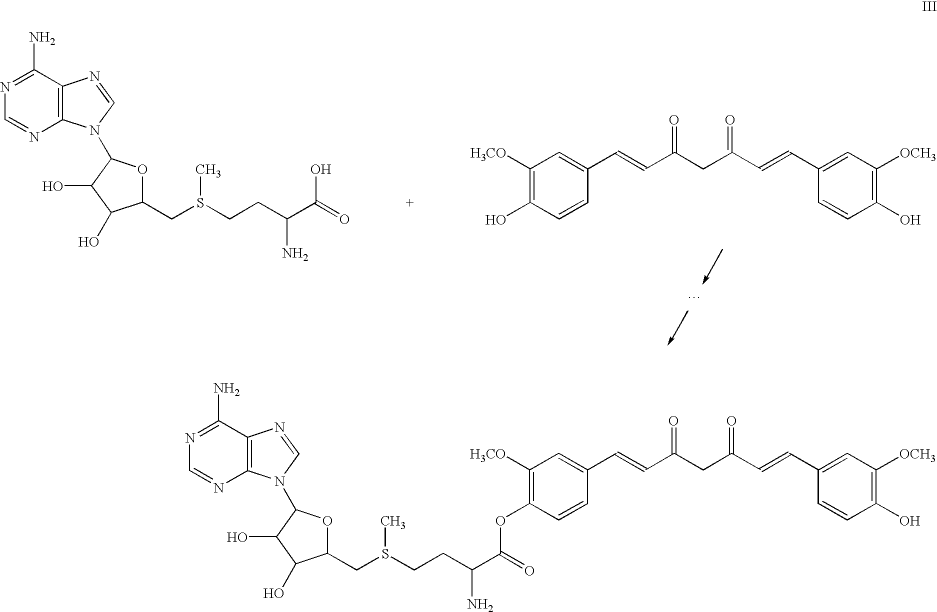

Where the radical-forming non-SAM constituent possesses more than one —OH, NH₂, or —COOH group which can be used to generate more than one radical to be linked to SAM, then each and every possible radical is within the scope of the present invention. The product and intermediates may be synthesized and isolated or purified employing one or more standard protecting/deprotecting steps and purification techniques as discussed above.

### SCHEME IV

As depicted by Scheme IV, stable salts of S-adenosylmethionine may be synthesized as described below:

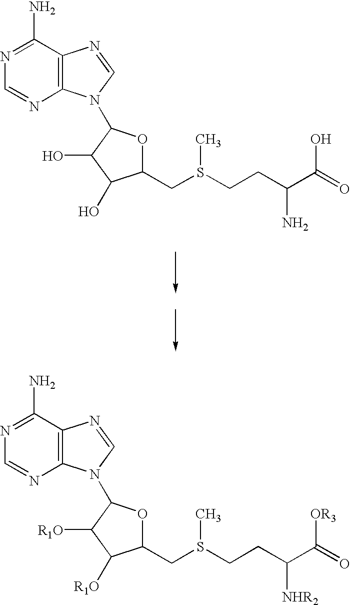

As described by the methods herein incorporated by reference, salts of SAM may be formed independently at each of R₁, R₂ or R₃. For example, one or more of R₁, R₂ or R₃ can remain as hydrogen while the other R groups may be formed as salts. The synthetic methods for producing stable salts (see U.S. Pat. Nos. 4,558,122 and 5,166,328) are known in the art. U.S. Pat. Nos. 4,558,122 and 5,166,328 are incorporated herein by reference as though set forth in full.

Stable salts of SAM as described by Scheme IV can optionally be used in place of S-adenosylmethionine in Schemes I, II, and III to synthesize stable salts of compounds of formula I.

### Route(s) of Administration

The route(s) of administration of the compounds and compositions of the present invention are well known to those skilled in the art (see, for example, “Remington's Pharmaceutical Sciences”, 18th Edition, Chapter 86, pp. 1581-1592, Mack Publishing Company, 1990). The compounds and compositions may be administered orally, parenterally, by inhalation spray, topically, rectally, nasally, buccally, vaginally, or via an implanted reservoir in dosage formulations containing conventional non-toxic pharmaceutically-acceptable carriers, adjuvants, and vehicles. The term parenteral as used herein includes subcutaneous, intravenous, intramuscular, intraperitoneally, intrathecally, intraventricularly, intrasternal, and intracranial injection or infusion techniques.

To be effective therapeutically as central nervous system targets, the compounds and compositions should readily penetrate the blood-brain barrier when peripherally administered. Compounds which cannot penetrate the blood-brain barrier can be effectively administered by an intraventricular route.

The compounds and compositions may be administered in the form of sterile injectable preparations, for example, as sterile injectable aqueous or oleaginous suspensions. These suspensions, may be formulated according to techniques known in the art using suitable dispersing or wetting agents and suspending agents. The sterile injectable preparations may also be sterile injectable solutions or suspensions in non-toxic parenterally-acceptable diluents or solvents, for example, as solutions in 1,3-butanediol. Among the acceptable vehicles and solvents that may be employed are water, Ringer's solution and isotonic sodium chloride solution. In addition, sterile, fixed oils are conventionally employed as solvents or suspending mediums. For this purpose, any bland fixed oil such as a synthetic mono- or di-glyceride may be employed. Fatty acids such as oleic acid and its glyceride derivatives, including olive oil and castor oil, especially in their polyoxyethylated versions, are useful in the preparation of injectables. These oil solutions or suspensions may also contain long-chain alcohol diluents or dispersants.

Additionally, in a preferred embodiment, the compounds and compositions may be administered orally in the form of capsules, tablets, aqueous suspensions, or solutions. Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient. The oral dosage forms may further contain sweetening, flavoring, coloring agents, or combinations thereof. Delivery in an enterically coated tablet, caplet, or capsule, to further enhance stability and provide release in the intestinal tract to improve absorption, is the best mode of administration currently contemplated.

The compounds may also be administered rectally in the form of suppositories. These compositions can be prepared by mixing the drug with a suitable non-irritating excipient which is solid at room temperature, but liquid at rectal temperature and, therefore, will melt in the rectum to release the drug. Such materials include cocoa butter, beeswax, and polyethylene glycols.

Furthermore, the compounds may be administered topically, especially when the conditions addressed for treatment involve areas or organs readily accessible by topical application, including the lower intestinal tract. Suitable topical formulations can be readily prepared for such areas or organs. For example, topical application to the lower intestinal tract can be effected in a rectal suppository formulations (see above) or in suitable enema formulations.

It is envisioned that the continuous administration or sustained delivery of the compounds and compositions of the present invention may be advantageous for a given condition. While continuous administration may be accomplished via a mechanical means, such as with an infusion pump, it is contemplated that other modes of continuous or near continuous administration may be practiced. For example, such administration may be by subcutaneous or muscular injections as well as oral pills.

Techniques for formulating a variety of other sustained- or controlled-delivery means, such as liposome carriers, bio-erodible particles or beads and depot injections, are also known to those skilled in the art.

### Dosage

Dosage levels on the order of about 0.001 mg to about 100 mg per kilogram body weight of the active ingredient compounds or compositions are useful in the treatment of the above conditions, with preferred levels ranging from 2.0 mg to 16 mg per day per kilogram body weight. The compounds and compositions of the present invention may usually be given in two or three doses daily. Starting with a low dose (200-300 mg) twice daily and slowly working up to higher doses if needed is a preferred strategy. The amount of active ingredient that may be combined with the carrier materials to produce a single dosage form will vary depending upon the host treated and the particular mode of administration.

It is understood, however, that a specific dose level for any particular patient will depend upon a variety of factors, including the activity of the specific compound employed; the age, body weight, general health, sex and diet of the patient; the time of administration; the rate of excretion; drug combination; the severity of the particular disorder being treated; and the form of administration. One of ordinary skill in the art would appreciate the variability of such factors and would be able to establish specific dose levels using no more than routine experimentation.

### EXAMPLES

The following examples are illustrative of the present invention and are not intended to be limitations thereon. Unless otherwise indicated, all percentages are based upon 100% by weight of the final composition.

### Example 1

### Preparation of α-(S-adenosylmethionine)-O-tocopherol

N-Acetyl-S-benzyl-L-homocysteine (0.5 g) was dissolved in 1,4-dioxane (25 ml). Dicyclohexyl carbodiimide (0.5 g) was added to the solution with stirring followed by the addition of α-tocopherol (1 g). The resulting reaction mixture was stirred at 30°-32° C. for eighteen (18) hours during which time a white precipitate separated. The mixture was then filtered and the filtrate evaporated to dryness in vacuo to give an expected oily residue.

The oily residue was added to dry ammonia (ca. 100 ml) which had been previously condensed in a 500 ml three-necked flask equipped with a stirrer and sodium hydroxide tube to maintain anhydrous conditions. While stirring, sodium was added to the reaction mixture in small pieces until the resulting blue color persisted for 5-10 minutes.

5′-O-p-Tolyl-sulfonyladenosine (0.5 g) was then added to the solution and stirring continued for ten (10) minutes. The ammonia was evaporated for three hours and the final traces thereof removed under diminished pressure, yielding a waxy solid residue. The residue was extracted with methylene chloride (2×25 ml) and the combined residue evaporated to dryness to give a waxy solid.

The waxy solid was dissolved in dimethyl sulfoxide (10 ml) containing acetic acid (3 ml) and the solution stirred with excess methyl iodide (1 ml) for 30 hours at 30°-32° C. The solvent was allowed to evaporate and the resulting residue was extracted with methylene chloride (25 ml) and dried with sodium sulfate. Evaporation of the solvent gave a clear oil which turned green when exposed to air.

The expected compound, α-(S-adenosylmethionine)-O-tocopherol, was recovered and stored for future use.

### Example 2

A patient is suffering from depression. An S-adenosylmethionine derivative as identified above, or a pharmaceutical composition comprising the same, may be administered to the patient. Reduction or elimination of depression and mood enhancement are expected to occur following treatment.

### Example 3

A patient is suffering from a liver disease or disorder involving hepatic glutathione levels. An S-adenosylmethionine derivative as identified above, or a pharmaceutical composition comprising the same, may be administered to the patient. Enhancement of liver detoxification function is expected to occur following treatment.

### Example 4

A patient is suffering from impaired heart and/or artery function resulting from elevated blood levels of homocysteine. An S-adenosylmethionine derivative as identified above, or a pharmaceutical composition comprising the same, may be administered to the patient. Enhancement of heart and/or artery function, and/or reduction in heart and/or artery disease risk is expected to occur following treatment.

### Example 5

A patient is suffering from degenerative joint disease. An S-adenosylmethionine derivative as identified above, or a pharmaceutical composition comprising the same, may be administered to the patient. Stimulation of chondrocytes to increase production of new cartilage and enhancement of joint health, mobility, and comfort are expected to occur following treatment.

### Example 6

A patient is suffering from a disease or disease condition induced or exacerbated by cellular senescence. An S-adenosylmethionine derivative as identified above, or a pharmaceutical composition comprising the same, may be administered to the patient. Reduction or elimination of cellular senescence is expected to occur following treatment.

The invention being thus described, it will be obvious that the same may be varied in many ways. Such variations are not to be regarded as a departure from the spirit and scope of the invention and all such modifications are intended to be included within the scope of the following claims.

We claim:

1. A compound of formula I: 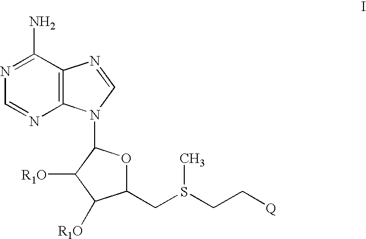
or a pharmaceutically acceptable salt, ester, or solvate, thereof, wherein:
R₁ is hydrogen, C₁-C₁₀ alkyl, C₂-C₁₀ alkenyl or alkynyl, or —C(O)R₂ where R₂ is C₁-C₁₀ alkyl or C₂-C₁₀ alkenyl or alkynyl;
Q is 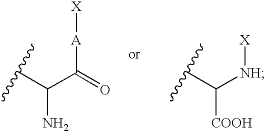
wherein
A is O or N; and
X is G, M, Y, or Z;
wherein G is a reaction product derived from a reactant compound selected from the group consisting of α-butyric acid, 3-methoxy-4-hydroxymandelic acid, 3-carboxy-3-aminopropyl analogues, e.g., wye base and diphthamide, 5-phosphoribose-1-pyrophosphoric acid, 6-gingerol, acetyl-L-carnitine, acetylcholine, ajoene, aminocyclopropane-carboxylic acid (ACC), anserine, anthocyanin, apigenin, arachidonic acid, astaxanthin, betaine, biopterin, calcium pectate, carbamyl phosphate, carnitine, carnosine, catechin, chlorogenic acid, choline, creatine, creatinine, cryptoxanthin, cumic acid, cumidine, curcumin, cyanidin chloride, d-limonene, daidzein, diacylglycerol, dopamine, ellagic acid, epicatechin, epicatechin gallate, epigallocatechin, epigallocatechin gallate, epinephrine, farnesyl, fibronectins, fisetin, (flavan-3-ol)ₙ, wherein n is 1-12, flavoxanthine, fructose 1,6-bisphosphate, gallic acid, genistein, geranyl, ginkgolide A, ginkgolide B, ginkgolide C, glucose, glutathione, GTP, hesperidin, hesperitin, histamine, HMG Co-A, homoserine lactone, indole-3-carbinol, kynurenine, L-dopa, L-histidine, linatine, lipoic acid, lupeol, lutein, luteolin, lycophyll, lycoxanthine, lysine, lysolecithin, mandelic acid, melanins, melatonin, metanephrine, methylated estrogen, methylated lipids, N-methylglycine, N-methyl histamine, N-malonyl ACC, neopterin, nervonic acid, N,N-dimethylglycine, N,N-dimethyltryptamine, norepinephrine, normetanephrine, ornithine, p-coumaric acid, pectin, phosphocreatine, phytic acid, phytochlorin, phytol, picolinic acid, proanthocyanin, pyruvate, quercetin, queuine, queuosine, quinolinic acid, rutin, S-allymercaptocysteine, sarcosine, serotonin, sesamin, silybin, sulphorane, taurine, taxicatin, taxicin I, taxicin II, taxifolin, taxine A, taxodione, tetrahydrobiopterin and derivatives, trimethylysine, tryptamine, tumeric, vaccenic acid, vanillic acid, xanthophyll, xanthoxylin, or zeaxanthin;
wherein M is a straight or branched C₁-C₁₁ alkyl, or C₂-C₁₁ straight or branched alkenyl or alkynyl, wherein said alkyl, alkenyl, and alkynyl is substituted with 1 to 8 substituents selected from the group consisting of hydroxy, carboxy, amino, and —SH, wherein said hydroxy, carboxy, amino, or —SH substituent is optionally substituted; M is further optionally substituted with 1 to 8 substituents selected from the group consisting of halo, nitro, double-bonded oxygen, methoxy, and C₂-C₈ straight or branched alkoxy; and wherein one or more carbon atom(s) of said alkyl, alkenyl, or alkynyl is optionally replaced with nitrogen, oxygen, or sulfur;
wherein Y is a straight or branched C₁₂-C₃₀ alkyl, alkenyl, or alkynyl optionally substituted with 1 to 12 substituents selected from the group consisting of hydroxy, carboxy, amino, halo, nitro, —SH, and J, wherein J is phenyl or a 5-7 membered O-heterocyclic ring, and J is optionally substituted with 1 to 5 substituents selected from the group consisting of hydroxy, carboxy, amino, halo, nitro, —SH, C₁-C₁₀ alkyl, C₂-C₁₀ alkenyl or alkynyl, methoxy, C₂-C₈ straight or branched alkoxy, and —OC(O)R₂ where R₂ is C₁-C₁₀ alkyl or C₂-C₁₀ alkenyl or alkynyl, wherein said hydroxy, carboxy, amino, halo, nitro, —SH, C₁-C₁₀ alkyl, C₂-C₁₀ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; and
wherein Z is phenyl substituted with 1 to 5 substituents selected from the group consisting of C₁-C₈ straight or branched alkyl, hydroxy, carboxy, amino, and —SH; and Z is further optionally substituted with 1 to 3 substituents selected from the group consisting of halo, nitro, C₁-C₈ straight or branched alkyl, C₂-C₈ straight or branched alkenyl or alkynyl, methoxy, and C₂-C₈ straight or branched alkoxy, wherein said hydroxy, carboxy, amino, —SH, halo, nitro, C₁-C₈ straight or branched alkyl, C₂-C₈ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur.

2. The compound of claim 1, wherein R₁ is hydrogen, methyl, or —C(O)CH₃.

3. The compound of claim 1, wherein A is oxygen.

4. The compound of claim 3, wherein X is Y and J is phenyl.

5. The compound of claim 4, wherein Q is (2,6-di(tert-butyl))4-methylphenol.

6. The compound of claim 1, wherein X is M.

7. The compound of claim 1, wherein X is Y.

8. The compound of claim 1, wherein X is Z.

9. A pharmaceutical composition comprising:
(i) a compound of formula (I): 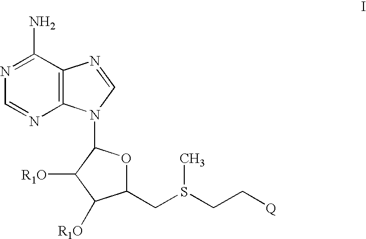
or a pharmaceutically acceptable salt, ester, or solvate, thereof, wherein:
R₁ is hydrogen, C₁-C₁₀ alkyl, C₂-C₁₀ alkenyl or alkynyl, or —C(O)R₂ where R₂ is C₁-C₁₀ alkyl or C₂-C₁₀ alkenyl or alkynyl;
Q is 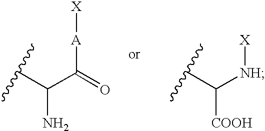
wherein
A is O or N; and
X is G, M, Y, or Z;
wherein G is a reaction product derived from a reactant compound selected from the group consisting of α-butyric acid, 3-methoxy-4-hydroxymandelic acid, 3-carboxy-3-aminopropyl analogues, e.g., wye base and diphthamide, 5-phosphoribose-1- pyrophosphoric acid, 6-gingerol, acetyl-L-carnitine, acetylcholine, ajoene, aminocyclopropane-carboxylic acid (ACC), anserine, anthocyanin, apigenin, arachidonic acid, astaxanthin, betaine, biopterin, calcium pectate, carbamyl phosphate, carnitine, carnosine, catechin, chlorogenic acid, choline, creatine, creatinine, cryptoxanthin, cumic acid, cumidine, curcumin, cyanidin chloride, d-limonene, daidzein, diacylglycerol, dopamine, ellagic acid, epicatechin, epicatechin gallate, epigallocatechin, epigallocatechin gallate, epinephrine, farnesyl, fibronectins, fisetin, (flavan-3-ol)ₙ, wherein n is 1-12, flavoxanthine, fructose 1,6-bisphosphate, gallic acid, genistein, geranyl, ginkgolide A, ginkgolide B, ginkgolide C, glucose, glutathione, GTP, hesperidin, hesperitin, histamine, HMG Co-A, homoserine lactone, indole-3-carbinol, kynurenine, L-dopa, L-histidine, linatine, lipoic acid, lupeol, lutein, luteolin, lycophyll, lycoxanthine, lysine, lysolecithin, mandelic acid, melanins, melatonin, metanephrine, methylated estrogen, methylated lipids, N-methylglycine, N-methyl histamine, N-malonyl ACC, neopterin, nervonic acid, N,N-dimethylglycine, N,N-dimethyltryptamine, norepinephrine, normetanephrine, ornithine, p-coumaric acid, pectin, phosphocreatine, phytic acid, phytochlorin, phytol, picolinic acid, proanthocyanin, pyruvate, quercetin, queuine, queuosine, quinolinic acid, rutin, S-allymercaptocysteine, sarcosine, serotonin, sesamin, silybin, sulphorane, taurine, taxicatin, taxicin I, taxicin II, taxifolin, taxine A, taxodione, tetrahydrobiopterin and derivatives, trimethylysine, tryptamine, tumeric, vaccenic acid, vanillic acid, xanthophyll, xanthoxylin, or zeaxanthin;
wherein M is a straight or branched C₁-C₁₁ alkyl, or C₂-C₁₁ straight or branched alkenyl or alkynyl, wherein said alkyl, alkenyl, and alkynyl is substituted with 1 to 8 substituents selected from the group consisting of hydroxy, carboxy, amino, and —SH, wherein said hydroxy, carboxy, amino, or —SH substituent is optionally substituted; M is further optionally substituted with 1 to 8 substituents selected from the group consisting of halo, nitro, double-bonded oxygen, methoxy, and C₂-C₈ straight or branched alkoxy; and wherein one or more carbon atom(s) of said alkyl, alkenyl, or alkynyl is optionally replaced with nitrogen, oxygen, or sulfur;
wherein Y is a straight or branched C₁₂-C₃₀ alkyl, alkenyl, or alkynyl optionally substituted with 1 to 12 substituents selected from the group consisting of hydroxy, carboxy, amino, halo, nitro, —SH, and J, wherein J is phenyl or a 5-7 membered O-heterocyclic ring, and J is optionally substituted with 1 to 5 substituents selected from the group consisting of hydroxy, carboxy, amino, halo, nitro, —SH, C₁-C₁₀ alkyl, C₂-C₁₀ alkenyl or alkynyl, methoxy, C₂-C₈ straight or branched alkoxy, and —OC(O)R₂ where R₂ is C₁-C₁₀ alkyl or C₂-C₁₀ alkenyl or alkynyl, wherein said hydroxy, carboxy, amino, halo, nitro, —SH, C₁-C₁₀ alkyl, C₂-C₁₁ alkenyl or alkynyl, methoxy, C₂-C₈ straight or branched alkoxy, and —OC(O)R₂ substituents are optionally substituted; and wherein one or more carbon atom(s) of said alkyl, alkenyl, alkynyl, or alkoxy, is optionally replaced with nitrogen, oxygen, or sulfur; and
wherein Z is phenyl substituted with 1 to 5 substituents selected from the group consisting of C₁-C₈ straight or branched alkyl, hydroxy, carboxy, amino, and —SH; and Z is further optionally substituted with 1 to 3 substituents selected from the group consisting of halo, nitro, C₁-C₈ straight or branched alkyl, C₂-C₈ straight or branched alkenyl or alkynyl, methoxy, and C₂-C₈ straight or branched alkoxy, wherein said hydroxy, carboxy, amino, —SH, halo, nitro, C₁-C₈ straight or branched alkyl, C₂-C₈ straight or branched alkenyl or alkynyl, methoxy, and C₂-C₈ straight or branched alkoxy substituents are optionally substituted; and wherein one or more carbon atom(s) of said alkyl, alkenyl, alkynyl, or alkoxy, is optionally replaced with nitrogen, oxygen, or sulfur; and
(ii) a pharmaceutically acceptable carrier.

10. The pharmaceutical composition of claim 9, wherein said compound methylates a target molecule in vitro.

11. The pharmaceutical composition of claim 10, wherein said target molecule is a protein, nucleic acid, lipid, glycoprotein, or glycolipid.

12. The pharmaceutical composition of claim 9, wherein the carrier is a sterile solution, suspension or emulsion, in a single or divided dose.

13. The pharmaceutical composition of claim 9, wherein the carrier is a capsule or tablet containing a single or divided dose of said compound.

14. The pharmaceutical composition of claim 9, wherein the carrier comprises a biodegradable polymer.

15. The pharmaceutical composition of claim 14, wherein the biodegradable polymer releases the compound of formula I over a prolonged period of time.

16. The pharmaceutical composition of claim 9, wherein the carrier is a solid implant.

17. A method for effecting a biological activity in an animal, which comprises administering to said animal an effective amount of a compound of formula I of claim 1, wherein the biological activity selected from the group consisting of neurochemical activity; liver biology activity; heart and artery function; cartilage, bone and joint health; stomach and/or intestinal lining resistance to ulceration; immune function; cell membrane integrity; and pain and inflammation; treating or preventing diseases or conditions; treating viral infections, infectious diseases, and leukemia; reducing the risk of Sudden Infant Death Syndrome; and control of obesity.

18. The method of claim 17, wherein the disease or condition is selected from the group consisting of tissue damage resulting from physical trauma, tissue damage resulting from cell damage or cell death due to necrosis or apoptosis, neuronal mediated tissue damage or diseases, neural tissue damage resulting from ischemia and reperfusion injury, neurological disorders and neurodegenerative diseases, vascular stroke, cardiovascular disorders, age-related macular degeneration, AIDS and other immune diseases, arthritis, atherosclerosis, cachexia, cancer, degenerative diseases of skeletal muscle involving replicative senescence, diabetes, head trauma, immune senescence, inflammatory bowel disorders, muscular dystrophy, osteoarthritis, osteoporosis, chronic pain, acute pain, neuropathic pain, nervous insult, peripheral nerve injury, renal failure, retinal ischemia, septic shock, skin aging, altered circadian rhythmicicty, obesity, sickle cell anemia, cystic fibrosis, diseases or disorders relating to lifespan or proliferative capacity of cells, and diseases or disease conditions induced or exacerbated by cellular senescence.

19. The method of claim 17, wherein said effect on neurochemical activity is selected from the group consisting of treating anxiety; treating depression; treating depression secondary to a chronic disease; treating dementia; treating schizophrenia; treating Alzheimer's disease; treating Parkinson's disease; treating demyelinating disorders; treating peripheral neuropathies; treating uremic neuropathy; treating Grand Mal seizures; treating Tay-Sachs disease; treating epilepsy; enhancing mood and behavior; and maintaining or effecting neuronal membrane ratios of phosphatidyl choline and cholesterol.

20. The method of claim 17, wherein said effect on liver biology activity is selected from the group consisting of treating cirrhosis, chronic liver disease, alcoholic liver damage, toxic chemical exposure, NSAID-liver damage, estrogen induced liver problems, bile disorders, and environmental chemical hypersensitivity.

21. The method of claim 17, wherein said effect on heart and artery function is treating or reducing heart and/or artery disease risk due to elevated blood levels of homocysteine.

22. The method of claim 17, wherein said effect on cartilage, bone and joint health is selected from the group consisting of treating osteoarthritis, rheumatoid arthritis, fibromyalgia, joint injuries, joint inflammation, joint degeneration, and osteoporosis.

23. The method of claim 17, wherein said effect on immune function is selected from the group consisting of treating organ transplant rejection, graft rejection, lupus, uveitis, Behcet's disease, Graves disease, Guillain-Barre syndrome, psoriasis, acute dermatomyositis, atopic skin disease, scleroderma, eczema, aplastic anemia, primary cirrhosis, autoimmune hepatitis, ulcerative colitis, Crohn's disease, amyotrophic lateral sclerosis, myasthenia gravis, multiple sclerosis, nephrotic syndrome, glomerulonephritis, rheumatoid arthritis, and diabetes mellitus.
